(19) United States
(12) Patent Application Publication
(10) Pub. No.: US 2006/0275233 A1
(43) Pub. Date: Dec. 7, 2006

(54) Long-lasting color and gloss formulations

(76) Inventors: Yoram Fishman, Los Angeles, CA (US); Tamara Rayter, Granada Hills, CA (US)

Correspondence Address: KONRAD RAYNES & VICTOR, LLP, 315 S. BEVERLY DRIVE, # 210, BEVERLY HILLS, CA, 90212, US

(21) Appl. No.: 11/401,642

(22) Filed: Apr. 11, 2006

Related U.S. Application Data
(63) Continuation-in-part of application No. 11/103,129, filed on Apr. 11, 2005.

(51) Int. Cl. A61K 8/81; A61K 8/97; A61K 8/72

(52) U.S. Cl. 424/64; 424/74; 424/70.31; 424/70.11

(57) ABSTRACT

Embodiments include colorant compositions that are water-based and gloss compositions. Certain liquid lip color embodiments include a composition having water and shellac. The composition may also include a neutralizer such as triethanolamine to neutralize the shellac. The composition may also include PPG-17/IPDI/DMPA copolymer. The composition may also include an emulsifier such as acrylates/acrylamide copolymer and mineral oil and polysorbate 85. In addition, the composition may also include a colorant. Embodiments also include gloss compositions including polybutene, polyethylene, and shea butter. Other ingredients may also be present in the liquid lip color and the gloss compositions. Other embodiments are described and claimed.

This is a Continuation-in-part of U.S. application Ser. No. 11/103,129, filed Apr. 11, 2005, which is hereby incorporated by reference in its entirety.

### FIELD OF THE INVENTION

Certain embodiments relate to long-lasting liquid color formulations and to gloss formulations for application to the skin and methods for manufacturing such formulations.

### DESCRIPTION OF THE RELATED ART

Colorant materials have been used for many years to highlight the lips and other skin regions on the body. Most conventional formulations include a colorant such as a pigment mixed with an oily vehicle such as a fat or oil stiffened to a desired consistency with one or more waxes. In recent years, attempts have been made to provide a lipstick and other cosmetics having a long lasting color, in order to lessen the need for frequent reapplication and to avoid problems such as the color rubbing off of the lips and onto clothing.

One type of formulation increases the concentration of the colorant in an effort to obtain long lasting color. However, increasing the concentration of the colorant can cause the formulation to become too dry on the lips. Moisturizers and conditioning agents can be added to the formulations, but these components are not durable and can adversely affect the appearance on the lips.

Another type of formulation includes a ceramide component. Ceramides are a group of lipids, members of which are found in the epidermis of mammals. Chemically, ceramides are N-acrylated sphingosine bases. Ceramide lipstick formulations also typically contain hydrocarbon waxes.

Certain liquid lip color compositions have also been developed. Such compositions have included substantial quantities of alcohol, which is undesirable for a number of reasons including stinging or irritating the lips or skin during and after application.

Colorants used for application to skin other than the lips, such as tattoos on the arms, may be injected into the skin in order to form a permanent tattoo. Other colorant formulations may be used to stain the skin for a relatively short time period, for example, a few hours. It would be desirable to be able to color the skin for a longer time period, while at the same time not permanently staining or irritating the skin.

### BRIEF DESCRIPTION OF THE DRAWINGS

Certain embodiments of the invention are described with reference to the accompanying drawing which, for illustrative purposes, are schematic and not drawn to scale.

FIG. 1 illustrates a cross-sectional view of a container that may be used for storing and applying compositions, in accordance with certain embodiments.

FIG. 2 illustrates a view of a band having a cut out design for use as a stencil that can be used when applying color compositions to skin.

### DESCRIPTION OF PREFERRED EMBODIMENTS

Embodiments of the present invention relate to skin coloring formulations having long-lasting qualities and easy application. Certain embodiments include water-based cosmetic compositions. Water-based cosmetics have a number of advantages over alcohol-based cosmetics. For example, by being water-based, the compositions are less likely to sting the user and less likely to have an unpleasant odor.

Certain preferred embodiments contain a shellac component, a neutralizer component, a water component, an emulsifier component, and a polymer comprising PPG-17/IDPI/DPMA copolymer (INCI name). It is believed that the combination of the shellac and the PPG-17/IDPI/DPMA copolymer acts to bind the formulation to the skin, yielding a long-lasting cosmetic.

The PPG-17/IDPI/DPMA copolymer is available under the trade name AVALURE UR 450, from Noveon, Inc. Certain embodiments may include approximately 5 to approximately 90 weight percent of the polymer, with certain preferred embodiments including approximately 20 to approximately 40 weight percent, and certain more preferred embodiments including approximately 30 to approximately 33 weight percent. Other embodiments may utilize a different polymer, for example, Polyurethane-4 (INCI name), which is available under the trade name AVALURE UR 445, from Noveon, Inc.

Shellac is also utilized in certain embodiments of the present invention. Shellac refers to all forms of purified lac, a natural resin secreted by the lac insect. Certain embodiments may include up to approximately 10 weight percent shellac, with certain preferred embodiments including approximately 1 to approximately 6 weight percent shellac, and certain more preferred embodiments including approximately 3 weight percent. Shellac is generally not soluble in cold and neutral pH water and as a result, in certain embodiments, it is desirable to have a pH of about 8 and a temperature of approximately 55° C. This may be accomplished by heating and using a neutralizer to solubilize the shellac in water. A variety of suitable neutralizers may be used to enable the shellac to be soluble in water, including, but not limited to, triethanolamine (TEA), aminoethylpropanol (AMP), sodium hydroxide, and potassium hydroxide. Certain embodiments utilize up to about 5 weight percent neutralizer, with certain preferred embodiments including up to about 2 weight percent, and certain more preferred embodiments including about 0.5 weight percent triethanolamine 85%.

Purified water is also utilized in certain embodiments of the present invention. Certain embodiments include approximately 10 to approximately 70 weight percent water, with certain preferred embodiments including approximately 30 to approximately 50 weight percent water, with certain more preferred embodiments including approximately 40 to approximately 50 weight percent water.

An emulsifier is also utilized in certain embodiments of the present invention. Certain preferred embodiments utilize a component having the INCI name Acrylates/Acrylamide Copolymer and Mineral Oil and Polysorbate 85, which is available under the trade name NOVEMER EC-1, from Noveon, Inc. Another emulsifier that may be used in certain embodiments is Acrylates/C10-30 Alkyl Acrylate Crosspolymer (INCI name), available under the trade name PEMULENE from Noveon. Certain embodiments include approximately 0.1 to 5 weight percent emulsifier, with more certain preferred embodiments including approximately 0.5 to 2 weight percent.

Certain embodiments also may utilize a surfactant. An example of a surfactant which may be used is sodium laureth sulfate (INCI name), available under the trade name SULFOCHEM ES-2K, from Noveon Inc. In certain embodiments, the surfactant is present in an amount of up to about 2 weight percent. The surfactant may be a combination of several different materials. For example, another surfactant which may be used is available under the trade name MIRACARE SLB 365, which is available from Rhodia, and which includes a number of different surfactants mixed together.

Certain embodiments may also include a film forming polymer such as Brassica Camprestis/Aleurites Fordi copolymer (INCI name), available under the trade name GLOSSAMER L6600, from Tri-K Industries, Inc.

Numerous dyes and pigments or color enhancing components may be utilized in lip color compositions according to certain embodiments of the present invention including, for example, U.S. Government certified colors, both Drug and Cosmetic grade and Food, Drug and Cosmetic grade. A colorant may include a variety of materials. Typical materials include various lakes, iron oxides, micas, and titanium dioxides. Some specific examples are sold under the following trade names or designations: PERMASHADE WP10S (Persperce Inc. of NJ); FLAMENCO Super Orange 330Z, FLAMENCO Super Violet 530Z, FLAMENCO Sparkle Violet 520J, FLAMENCO Super Red 430Z, (Mearl Corp., Ossining, N.Y.); D&C Red 33, FD&C Red 40, FD&C Yellow 6, D&C Red 7 Calcium Lake C19-031, (Sun Chemical Corp., Staten Island, N.Y.); Cosmetic Red Iron Oxide CG-180, Red Iron Oxide C33-W075, Dark Brown Iron Oxide CG975, (Whittaker, Clark & Daniels, South Plainfield, N.J.); CLOISONNE Super Rouge 450Z, CLOISONNE Sparkel Bronze 250J, (Engelhard Corp.); COLORONA Oriental Beige, COLORONA Sienna, COLORONA Super Red, COLORONA Aborigine Amber 017488 (Rona/EM Industries, Hawthorne, N.Y.); Numerous other colorants may also be used. Often more than one component is used to form the desired colorant and the total amount of colorant may in certain embodiments range up to approximately 20 weight percent or higher, with certain preferred embodiments having approximately 3-10 weight percent.

Depending on its texture, the colorant may be mixed with a liquid to form a slurry and then milled to break up the particles of pigment and obtain a product having better spreadability. Some examples of liquids which may preferably be mixed with the colorant prior to milling include an oil such as castor oil, glycerine, propylene glycol, butylene glycol, and various esters. A preferred liquid for mixing with the colorant is PPG-20 Methyl Glucose Ether (INCI name), available under the trade name GLUCAM P-20, from Noveon. In some embodiments approximately 2 parts of the PPG-20 Methyl Glucose Ether is mixed with 1 part colorant, although a variety of mixtures such as 1 to 1 and others are possible as long as a slurry is created. Certain embodiments may include up to approximately 10 weight percent methyl glucose ether, with more preferred embodiments including approximately 2 to approximately 4 weight percent. Generally, lakes, iron oxides, and titanium dioxides may be milled. Water soluble colorant materials may not need to be milled. For materials that are milled, a variety of mills, including a three roller mill manufactured by Exakt (OK), may be utilized.

In certain embodiments, the composition includes an additive component to improve its spreadability characteristics. This component may be a fatty alcohol such as, for example, isostearyl alcohol. Certain embodiments may include up to approximately 10 weight percent isostearyl alcohol, with preferred embodiments including up to approximately 5 weight percent and even more preferred embodiments including approximately 1 to approximately 3 weight percent. In other embodiments it may be possible to use fatty acids such as isostearyl acid to improve the spreadability of the colorant.

Certain embodiments may also include an additional ingredient for improving the feel of the lip color on the lips. Preferred embodiments utilize fine silica particles, which may be mixed with the colorant. It is also believed that the silica component contributes to the adhesion of the colorant to the skin. In certain embodiments the silica is available premixed with the colorant. Such a colorant with silica is titanium dioxide and silica, sold under the trade name PERMASHADE WP 10S. Certain embodiments may include up to approximately 5 weight percent PERMASHADE WP 10S. Fine particles of other materials, such as, for example, boron nitride, may also be used in other embodiments to improve the feel of the colorant on the skin.

Another ingredient which may be present in certain embodiments is lanolin oil, sold under the trade name LANTROL 1673. The lanolin oil may act to improve the feel of the composition and may promote adhesion with the shellac.

Another ingredient which may be present in certain embodiments is C10-30 Cholesterol/Lanosteral Esters (INCI name), available under the trade name SUPER STEROL LIQUID, (from Croda), which may act as a skin repair, conditioning and moisturizing agent for the lips, and may be present in certain liquid lip color embodiments at approximately 0.5 to 2 weight percent. Other embodiments may use up to approximately 10 weight percent, or higher.

Additional ingredients may also be added to certain embodiments of the present invention to enhance various aspects including preserving the formulation. Examples of preservatives which may be used include Methylparaben (INCI name), which may in certain embodiments be present up to about 1 wt %. Additionally, a number of commercially available preservative compositions include multiple preservative materials, for example, Propylene Glycol and Diazolidinyl Urea and Methylparaben and Propylparaben (INCI name), available under the trade name GERMABEN II, from International Specialty Products, which may in certain embodiments be present up to about 2 wt %. Another commercially available preservative includes a composition containing Propylene Glycol and Diazolidinyl Urea and Iodopropynyl Butylcarbamate (INCI name), available under the trade name LIQUID GERMALL PLUS, available from International Specialty Products, which may in certain embodiments be present up to about 1 wt %.

Another additive which may be present in certain embodiments is Tocopherol (INCI name), which is available under the trade name COVI-OX T-70. The tocopherol may be categorized as an anti-oxidant and in certain preferred embodiments may be present up to about 1 wt %. In addition, a sunscreen component such as octylmethoxycinnamate may be added if desired. Certain embodiments may include up to 7.5% octylmethoxycinnamate, with certain more preferred embodiments including approximately 1 weight percent octylmethoxycinnamate.

Certain embodiments of the present invention also relate to methods for forming liquid cosmetic compositions including liquid lip color. Several examples of methods are set forth below.

One method for manufacturing the Latte liquid lip color listed in Table 2 below is as follows. In a first container, the water is heated to about 55° C. and mixed with the methylparaben and the D&C Red 33 powder. The batch is mixed well and then the triethanolamine and shellac are added. In a separate container the isostearyl alcohol, the GLOSSAMER L6600, the lanolin oil (LANTROL 1673), and the NOVEMER EC1 are premixed and then added to the batch while mixing to emulsify the batch. The batch is then cooled to about 40° C. or a little below 40° C. and the AVALURE UR 450, GERBAMEN II, sodium laureth sulfate and COVI-OX T-70 are added and mixed well. Then the PERMASHADE WP 10S and GLUCAM P-20 are added to the batch while mixing. Then the dark brown iron oxide CG 975 and GLUCAM P-20 are added to the batch while mixing. Then the sparkle bronze 250J is added and the batch mixed well. After mixing the viscosity of the batch may be checked. If necessary, a small amount of NOVEMER EC1, such as 0.3 weight percent, is added to adjust the viscosity.

Another method for forming a liquid composition such as a lip color is as follows. In a first container, water is heated to about 55° C., and triethanolamine is added. The solution is mixed well and shellac is sprinkled in and mixed well until the shellac is dissolved. At this time the solution is preferably clear. Start cooling the batch. The oil phase ingredients are then added one by one, ending with the emulsifier (such as NOVEMER EC1) and the batch may then be emulsified for about 30 minutes. When the batch is cooled to about 40° C., the AVALURE UR 450 and colorants and preservatives are added and the batch mixed. If multiple preservatives are used, then it may in certain embodiments be desirable to mix them together prior to adding to the batch. It should be noted that certain elements of the method may be modified. For example, water soluble colorants may be added earlier in the process if desired. Additional additives may also be added if desired. For example, a sunscreen component such as octylmethoxycinnamate may be added.

Another method for forming a composition such as a liquid lip color is as follows. In a first container, AVALURE UR 450 is well mixed with milled colorants (which in certain embodiments may be mixed with PPG-20 Methyl Glucose Ether or may be in powder form) at room temperature. In a second container, water and neutralizer (for example, TEA) are mixed and heated to about 55-60° C., then the shellac is added and mixed until the solution is clear. Then other water soluble components including, for example, preservatives (for example, LIQUID GERMALL PLUS) and any FD&C colorants, are added to the second container solution and mixed. Then the second container solution is cooled to room temperature and slowly added to and mixed with the first container batch. In a third container, the oil phase ingredients (for example, isostearyl alcohol, GLOSSAMER L6600, lanolin oil, NOVEMER EC1, and C10-30 cholesterol/lanosteral esters) are mixed together at room temperature and then the mixed third container solution is mixed into the batch including the first and second container solutions. Then, the sodium laureth sulfate is added to the mixed batch. If desired, additional additives may also be included. For example, an additive such as tocopherol may be added to the oil phase ingredients in the third container prior to mixing the third container ingredients with the rest of the batch.

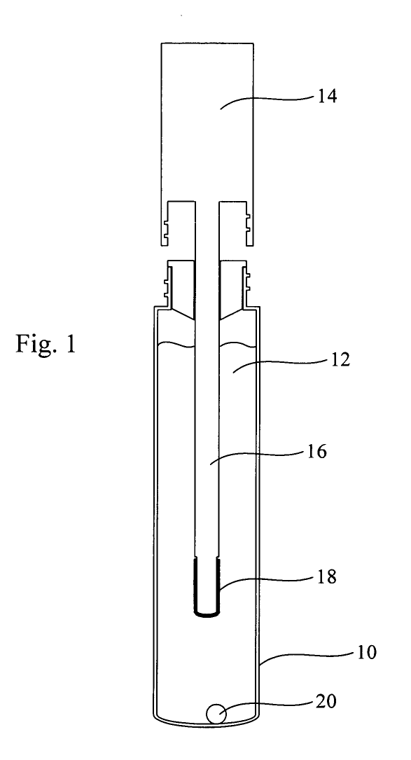

The cosmetic mixture (such as the latte lip color) may then be packaged, for example, in a tubular container having an applicator (or wand) with a tip brush or pad to apply to the lips or other skin. One example of a container and applicator is illustrated in FIG. 1, which is a cross-sectional view of a vial 10 containing a liquid lip color formulation according to embodiments of the present invention. Also included is a cap 14 having an integrated wand 16 and tip 18 for applying the liquid 12 to the lips. The container and applicator illustrated in FIG. 1 are available under the product name Mini Lip Gloss Container by World Wide Packaging (East Hanover, N.Y.). One or more small balls 20 (for example, stainless steel balls) may also be provided in the vial 10 for mixing the lip color 12. Other types of containers and applicators such as, for example, a pad or roller, may also be used.

Examples of liquid lip color compositions for achieving various colors according to certain embodiments of the present invention are set forth in Tables 1 and 2 below. Since there may be many modifications without departing from the scope of the invention, the examples below are not intended to limit the invention but to illustrate certain aspects of the invention more clearly.

TABLE 1
Examples of brown, hot pink, rose and flame color formulations.

| Ingredient (trade name) | Brown | | Hot Pink | | Rose | | Flame | |
|---|---|---|---|---|---|---|---|---|
| Purified Water | 45.540 | wt % | 40.95 | wt % | 40.95 | wt % | 40.950 | wt % |
| Methylparaben | — | | 0.10 | | 0.10 | | 0.100 | |
| Triethanolamine 85% | 0.550 | | 0.55 | | 0.55 | | 0.550 | |
| Shellac | 3.000 | | 3.00 | | 3.00 | | 3.000 | |
| Isostearyl Alcohol | 1.000 | | 2.50 | | 2.50 | | 2.500 | |
| GLOSSAMER L6600 | 6.500 | | 6.50 | | 6.50 | | 6.500 | |
| LANTROL 1673 | 0.500 | | 1.50 | | 1.50 | | 1.500 | |
| NOVEMER EC1 | 1.500 | | 2.00 | | 2.00 | | 2.000 | |
| AVALURE UR 450 | 30.450 | | 31.30 | | 31.30 | | 31.300 | |
| GERMABEN II | — | | 1.00 | | 1.00 | | 1.000 | |
| LIQUID GERMALL PLUS | 0.500 | | — | | — | | — | |
| SULFOCHEM ES-2K | 1.100 | | 1.00 | | 1.00 | | 1.000 | |
| COVI-OX T-70 | — | | 0.20 | | 0.20 | | 0.200 | |
| C10-30 Cholesterol/Lanosteral Esters | 1.000 | | — | | — | | — | |
| PERMASHADE WP 10S - | 1.200 | | 2.60 | | 2.50 | | 1.750 | |
| GLUCAM P-20 - | 3.625 | | 3.93 | | 2.90 | | 2.975 | |
| Cloisonne Super Rouge 450Z | — | | 1.00 | | — | | — | |
| D&C Red 7 Calcium Lake C19-031 | — | | 0.67 | | 0.20 | | — | |
| D&C Red 33 | — | | 0.15 | | 0.10 | | 0.180 | |
| Flamenco Super Red 430Z | — | | 1.05 | | — | | 3.000 | |
| FD&C Red 40 | 0.100 | | — | | 0.03 | | 0.270 | |
| FD&C Yellow 6 | 0.010 | | — | | — | | — | |
| Colorona Oriental Beige | 1.000 | | — | | — | | — | |
| Flamenco Sparkle Violet 520J | — | | — | | 3.67 | | — | |
| Cosmetic Red Iron Oxide CG180 | 2.425 | | — | | — | | 1.225 | |
| | 100.00 | | 100.000 | | 100.00 | | 100.000 | |

TABLE 2
Examples of ruby, latte, and raisin color formulations.

| Ingredient (trade name) | Ruby | | Latte | | Raisin | |
|---|---|---|---|---|---|---|
| Purified Water | 40.95 | (wt %) | 41.118 | (wt %) | 40.95 | (wt %) |
| Methylparaben | 0.10 | | 0.100 | | 0.10 | |
| Triethanolamine 85% | 0.55 | | 0.550 | | 0.55 | |
| Shellac | 3.00 | | 3.000 | | 3.00 | |
| Isostearyl Alcohol | 2.50 | | 2.500 | | 2.50 | |
| GLOSSAMER L6600 | 6.50 | | 6.500 | | 6.50 | |
| LANTROL 1673 | 1.50 | | 1.500 | | 1.50 | |
| NOVEMER EC1 | 2.00 | | 1.800 | | 2.00 | |
| AVALURE UR 450 | 31.30 | | 31.300 | | 31.30 | |
| GERMABEN II | 1.00 | | 1.000 | | 1.00 | |
| SULFOCHEM ES-2K | 1.00 | | 1.000 | | 1.00 | |
| COVI-OX T-70 | 0.20 | | 0.200 | | 0.20 | |
| PERMASHADE WP 10S | 1.00 | | 1.900 | | 0.50 | |
| GLUCAM P-20 | 2.60 | | 2.500 | | 2.45 | |
| Cosmetic Red Oxide CG180 | 1.60 | | — | | 1.40 | |
| FD&C Red 40 | 0.10 | | — | | — | |
| D&C Red 33 | 0.06 | | 0.032 | | — | |
| Colorona Sienna | 2.00 | | — | | — | |
| Flamenco Super | 2.04 | | — | | — | |
| Orange 330Z | | | | | | |
| Dark Brown Iron Oxide | — | | 0.600 | | — | |
| CG 975 | | | | | | |
| Aborgine Amber 017488 | — | | — | | 2.10 | |
| Red Iron Oxide C33-W075 | — | | — | | 0.55 | |
| Sparkle Bronze 250J | — | | 4.400 | | 2.40 | |
| | 100.00 | | 100.000 | | 100.00 | |

Certain embodiments relate to a gloss which may be used with lip color embodiments or by itself The gloss component may be applied on the lips in order to enhance the shine and texture of the lip color on the lips. The gloss composition may in certain embodiments be applied after lip color has been placed on the lips. Gloss embodiments may include polybutene, which may act to promote the adhesion of the gloss to the lips. Gloss embodiments may also include polyethylene (available from Jeen International Corp.), which may act as a binder and/or viscosity adjuster. Gloss embodiments may also include shea butter (INCI name Butyrospermum Parkii, available from Cognis), which may act as a moisturizer. One or more preservatives may also be present, for example, oil soluble preservatives, including, but not limited to, propyl paraben, and caprylyl glycol (available under the trade name LEXGARD 0, from Inolex Chem. Co.). Other additives such as C10-30 cholesterol/lanosteral esters (available as SUPER STEROL LIQUID, from Croda), which may act as a skin repair, conditioning and moisturizing agent, may also be present. Other additives such as colorants, glitter, etc, may also be present.

Certain embodiments include a base gloss composition including at least three of the materials listed above (polybutene, polyethylene, and shea butter). Certain embodiments may include a composition containing 20-90 weight percent polybutene, 0.5-10 weight percent polyethylene, 5-50 weight percent shea butter, 0-90 weight percent C10-30 cholesterol/lanosteral esters, and up to about 2 weight percent preservative. Certain more preferred embodiments include 40-60 weight percent polybutene, 1-3 weight percent polyethylene, 15-35 weight percent shea butter, 10-30 weight percent C10-30 cholesterol/lanosteral esters, and up to 1 weight percent preservative. The gloss may also in certain embodiments contain a colorant, which may include one or more components including, but not limited to, various micas, lakes, and other materials. The gloss may also include other additives if desired, for example, a sunscreen component such as octylmethoxycinnamate, present in quantities of up to approximately 5 weight percent.

One example of a gloss embodiment is set forth in Table 3 below.

TABLE 3
Lip gloss composition.

| Component | Weight Percent |
|---|---|
| Polybutene | 54.30 |
| Polyethylene | 1.90 |
| Butyrospermum Parkii (Shea Butter) | 25.00 |
| Propylparaben | 0.10 |
| C10-30 Cholesterol/Lanosterol Esters | 18.70 |

Embodiments also relate to methods for forming the gloss. In certain embodiments, polybutene is heated to about 65-70° C. The polyethylene is added and the heat may be stopped. The batch is mixed until the polyethylene melts. Then the shea butter added and the batch mixed well (the batch should still be warm enough to melt the shea butter). Then the preservative (for example, propylparaben) is added and the batch mixed well. Then the C10-30 cholesterol/lanosteral esters is added and the batch mixed well. If desired, the final batch can be transferred to final containers at an elevated temperature (for example, about 50° C.), when the viscosity is lower, because as the temperature decreases to room temperature, the product becomes more viscous and may be more difficult to package.

The gloss may be packaged in a variety of containers including, but not limited to, a container such as that illustrated in FIG. 1. A multi-component kit including, for example, a first container of lip color component and a second container of a lip gloss component, may also be prepared.

Another aspect of certain embodiments of the present invention relates to formulations for applying long-lasting but not permanent color to skin on the body other than the lips. Such other applications may include, but are not limited to, cosmetic applications such as foundation, eye liner, mascara, and tattoos. The lips may be more sensitive than other skin regions, and therefore certain ingredients and their concentrations may differ between lip color formulations and other skin embodiments. For example, tattoo embodiments may in certain embodiments employ a higher percentage of colorant than lip color formulations. Other tattoo embodiments may have the same compositions as set forth in Tables 1 and 2 above. Such embodiments will be generally temporary in nature. By temporary it is meant that the tattoo is not permanent in the sense of traditional tattoos in which a needle is inserted into through the skin and a colorant injected to form a permanent image.

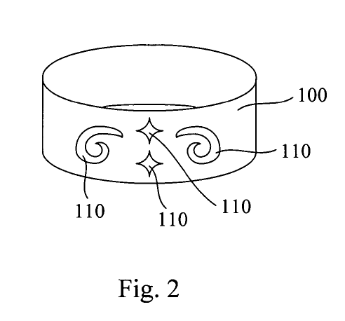

Various formulations may be applied to the skin using a variety of applicators. A roll-on applicator or a wand applicator (such as that illustrated in FIG. 1) may be used. One preferred applicator for applying a tattoo embodiment is a spray bottle or can. A stencil a may also be utilized together with liquid tattoo formulations. The stencil may be placed on the appropriate location on the skin, and the liquid tattoo formulation applied over the stencil. One example of a stencil includes a cut out design and a temporary adhesive for sticking the stencil on the skin. Another stencil may include, for example, a band that has a cut out design incorporated therein, such as the band 100 illustrated in FIG. 2, which includes cut out regions 110 through which a liquid formulation (such as, for example, one of the formulations shown in Tables 1 and 2) will be applied. If desired the cut out regions 110 may extend around the entire band 100. The band 100 may fit over or around a portion of the body such as, for example, an arm or leg. The band may be elastic or may have some other attachment mechanism (clip, velcro, etc.) for fitting around any portion of the body.

It will, of course, be understood that modifications of the present invention, in its various aspects, will be apparent to those skilled in the art. Other embodiments are also possible, their specific features depending upon the particular application. Therefore, the scope of the invention should not be limited by the particular embodiments herein described but should be defined by the appended claims and equivalents thereof.

What is claimed:

1. A cosmetic comprising effective amounts of:
water;
shellac;
a neutralizer adapted to neutralize the shellac;
PPG-17/IPDI/DMPA copolymer;
an emulsifier; and
C10-30 cholesterol/lanosteral esters.

2. The cosmetic of claim 1, comprising:
30-50 weight percent water;
1-10 weight percent shellac;
0.1-5.0 weight percent neutralizer
20-40 weight percent PPG-17/IPDI/DMPA copolymer;
0.5 to 5.0 weight percent emulsifier;
0.5 to 2.0 weight percent C10-30 cholesterol/lanosteral esters, and
0.1 to 15 weight percent colorant.

3. The cosmetic of claim 2, wherein the neutralizer is selected from at least one of the group consisting of triethanolamine, sodium hydroxide, and ammonium hydroxide; and
wherein the emulsifier is selected from at least one of the group consisting of (1) acrylates/acrylamide copolymer and mineral oil and polysorbate 85, and (2) acrylates/C10-30 alkyl acrylate crosspolymer.

4. The cosmetic of claim 3, wherein the cosmetic further comprises isostearyl alcohol present in an amount from 1-5 weight percent.

5. The cosmetic of claim 4, wherein the cosmetic further comprises brassica camprestis/aleurites fordi copolymer present in an amount from 1 to 12 weight percent.

6. The cosmetic of claim 5, wherein the cosmetic further comprises at least one ingredient selected from the group consisting of sodium laureth sulfate, lanolin oil, silica, and octylmethoxycinnamate.

7. A lip gloss composition, the lip gloss composition comprising effective amounts of polybutene, polyethylene, and shea butter.

8. The lip gloss composition of claim 7, wherein the lip gloss composition further comprises at least one ingredient selected from the group consisting of: (i) a preservative, and (ii) C10-30 cholesterol/lanosteral esters.

9. The lip gloss composition of claim 7, comprising:
40-60 weight percent polybutene;
1-3 weight percent polyethylene;
15-35 weight percent shea butter;
10-30 weight percent C10-30 cholesterol/lanosteral esters; and
up to 1 weight percent preservative.

10. The lip gloss composition of claim 9, further comprising a colorant.

11. A kit comprising:
a first container including a water-based liquid color formulation comprising effective amounts of water, shellac, a neutralizer adapted to neutralize the shellac, PPG-17/IPDI/DMPA copolymer, an emulsifier; and a colorant; and
a second container including a gloss formulation comprising effective amounts of polybutene, polyethylene, and shea butter.

12. The kit of claim 11, wherein the water-based liquid color formulation comprises:
10-70 weight percent water;
1-10 weight percent shellac;
0.1-5.0 weight percent neutralizer
5-90 weight percent PPG-17/IPDI/DMPA copolymer;
0.5 to 5.0 weight percent emulsifier; and
0.1 to 15 weight percent colorant.

13. The kit of claim 12, wherein the water-based liquid color formulation comprises 30-50 weight percent water and 20-40 weight percent PPG-17/IPDI/DMPA copolymer.

14. The kit of claim 13, wherein the neutralizer is selected from at least one of the group consisting of triethanolamine, sodium hydroxide, and ammonium hydroxide; and
wherein the emulsifier is selected from at least one of the group consisting of (1) acrylates/acrylamide copolymer and mineral oil and polysorbate 85, and (2) acrylates/C10-30 alkyl acrylate crosspolymer.

15. The kit of claim 13, wherein the water-based liquid color formulation further comprises isostearyl alcohol present in an amount from 1-5 weight percent.

16. The kit of claim 13, wherein the water-based liquid color formulation further comprises brassica camprestis/aleurites fordi copolymer present in an amount from 1 to 12 weight percent.

17. The kit of claim 13, wherein the water-based liquid color formulation further comprises at least one ingredient selected from the group consisting of sodium laureth sulfate, lanolin oil, silica, and octylmethoxycinnamate.

18. The kit of claim 13, wherein the lip gloss formulation further includes at least one ingredient selected from the group consisting of: (i) a preservative, and (ii) C10-30 cholesterol/lanosteral esters.

19. The kit of claim 13, wherein the lip gloss formulation comprises:
40-60 weight percent polybutene;
1-3 weight percent polyethylene;
15-35 weight percent shea butter;
15-25 weight percent C10-30 cholesterol/lanosteral esters; and
up to 1 weight percent preservative.

20. The kit of claim 13, wherein the lip gloss formulation includes a colorant.
